(19) United States
(12) Patent Application Publication
(10) Pub. No.: US 2020/0368175 A1
(43) Pub. Date: Nov. 26, 2020

(54) METHODS AND DEVICES FOR TREATING LEVODOPA INDUCED DYSKINESIA

(71) Applicant: CHRONO THERAPEUTICS INC., Hayward, CA (US)

(72) Inventors: Anubhav ARORA, Hayward, CA (US); Navdeep KAUR, San Jose, CA (US); Guy DiPIERRO, San Francisco, CA (US); Patrick H. RUANE, Dublin, CA (US); Ravinder D. PAMNANI, Los Altos, CA (US)

(21) Appl. No.: 16/474,083

(22) PCT Filed: Jan. 5, 2018
(86) PCT No.: PCT/US18/12646

§ 371 Date: Jun. 27, 2019

Related U.S. Application Data
(60) Provisional application No. 62/443,549, filed on Jan. 6, 2017.

(51) Int. Cl. A61K 9/70; A61K 31/465

(52) U.S. Cl. CPC A61K 9/7023; A61K 45/06; A61K 31/465

(57) ABSTRACT

A method of delivering nicotine to treat dyskinesia, such as Levodopa induced dyskinesia (LID), includes delivering a first dose of nicotine to a patient using a transdermal delivery device and delivering a second dose of nicotine to the patient using the transdermal delivery device. The first and second doses are timed such that a refractory period between peak plasma levels of nicotine in the patient prevents desensitization of nicotinic acetylcholine receptors while reducing symptoms of LID.

### CROSS-REFERENCE TO RELATED APPLICATIONS

This application is a U.S. National Phase Application Under 35 U.S.C. § 371 of International Application No. PCT/US2018/012646, filed on Jan. 5, 2018, titled “METHODS AND DEVICES FOR TREATING LEVODOPA INDUCED DYSKINESIA”, which claims priority to U.S. Provisional Application No. 62/443,549, filed Jan. 6, 2017, titled “METHODS AND DEVICES FOR TREATING LEVODOPA INDUCED DYSKINESIA”, the entirety of which is incorporated by reference herein.

### INCORPORATION BY REFERENCE

All publications and patent applications mentioned in this specification are incorporated herein by reference in their entirety to the same extent as if each individual publication or patent application was specifically and individually indicated to be incorporated by reference.

### FIELD

The present application relates generally to devices and methods for providing drug formulations or bioactive agents to a user, methodologies, and systems for individualization and optimization of drug delivery profiles, and companion applications through sensor-based and other patient-gathered data, to treat Parkinson's disease (PD) and levodopa induced dyskinesia (LID). More particularly, described herein are devices, compositions, and methods utilizing nicotine to reduce or eliminate one or more side effects associated with dopaminergic agent treatment. In some embodiments, the invention provides devices, compositions, and methods utilizing a combination of dopaminergic agents and other agents, such as such as amantadine in combination with nicotine, to reduce or eliminate one or more side effects associated with dopaminergic agent treatment.

### BACKGROUND

Some medicinal drugs are rapidly metabolized by the body. Multiple patient-individualized doses of the drug over a period of time are therefore often needed to provide a desired effect. In addition to having desired preventative or therapeutic effects, medicinal drugs can also have negative side effects on the body that can range from irritating to life-threatening. This is particularly true, for example, when titrating patients up to therapeutically effective amounts of nicotine transdermally. A person's body can also develop tolerance to a drug and therefore experience a diminished response to the drug after taking it for a period of time, thereby requiring higher doses to have an effect, and resulting in increased drug use and additional side-effects.

More particularly, many of the leading treatments for diseases lead to undesired side effects. For instance, levodopa, the standard treatment for Parkinson's disease (PD) treatment, is associated with debilitating abnormal involuntary movements or dyskinesia. Levodopa-induced dyskinesia (LID) often involves hyperkinetic movements, including chorea, dystonia, and athetosis. Few treatments are available for LID, possibly because the mechanisms responsible for its development are still uncertain. Although extensive studies have implicated numerous neurotransmitters, the cholinergic system has received little attention to date. This is somewhat surprising given the overlapping network of dopaminergic terminals and cholinergic interneurons in the striatum, and the well-known ability of nicotinic receptors to regulate striatal dopamine release. These motor abnormalities may occur after only a few months of treatment and affect the majority of patients taking levodopa within 5-20 years. Levodopa induced dyskinesia (LID) can be incapacitating, and it represents a major complication in Parkinson's disease management.

Parkinson's disease and associated diseases do not have an undisputed definition. This is connected with the fact that the cause is still unknown. Parkinson's disease is diagnosed by a battery of clinical syndromes, including motor signs (tremor at rest, rigidity, hypokinesia, and postural instability) and neuropsychological deficits that can even affect certain cognitive functions. Receptors play a significant role in Parkinson's disease, as they are the site for action of the dopamine liberated by the pre-synaptic element. D1 receptors are preferentially stimulated by dopamine. They are located on the post-synaptic membrane and are coupled to the adenylate-cyclase activity. They are localized in the striatum, the nucleus accumbens, and the olfactory tubercle. D2 receptors are preferentially stimulated by certain dopaminergic agonists such as bromocriptine and pyribedil.

A number of studies carried out both in man and in animals have demonstrated that these problems are, in large part, due to chronic degeneration of dopaminergic neurons of the nigrostriatal system. The current treatment, which remains the reference treatment, is treatment with levodopa accompanied, if necessary, by D2 receptor agonists, such as those cited above. However, as noted above, that type of treatment has medium to long-term side effects such as dyskinesia.

Parkinson's disease is common amongst those over 65, an age group that, in North America, is predicted to rise from 12% to 24% over the next 30 year. Further, the overall prevalence of Parkinson's disease in those over 65 population is on the order of 1.5-2% and increases with age. The risk of developing dyskinesia is even higher in patients who develop Parkinson's disease at an earlier age. In one published study, the incidence of developing dyskinesia was found to be 50% for patients in the age range of 40-59 years as compared to 16% for patients above 70 years of age. See Kumar. N. et al. Mov Disord. 2005 March; 20(3):342-4. Therefore, additional treatments are needed Parkinson's and LID.

Research has firmly established the connection between smoking and the reduced risk of Parkinson's disease, generally showing that active smokers have the lowest Parkinson's risk, followed by former smokers, while people who have never smoked have the highest risk. Indeed, a study published in the May 2015 issue of the American Journal of Epidemiology reported that people with a history of smoking had a 45% lower risk of developing Parkinson's. Other research has shown a similar level of risk, including a large National Institutes of Health study reported in Neurology in 2010, which found that current smokers had a 44% lower risk of Parkinson's than people who had never smoked. The findings further showed that past smokers had a reduced Parkinson's risk that was inversely related to the number of years they had smoked. Compared with people who had never smoked, former smokers who had smoked for 30 years or more had a 41% lower risk, while those who had smoked for 20 to 29 years had a 36% lower risk, and those who smoked for 10 to 19 years had a 22% lower risk.

It has been suggested that nicotine has the property of activating nicotinic cholinergic receptors on acute administration, causing an increase in the number of such receptors on chronic administration of nicotine to animals (D. J. K. Balfour et al., Pharmacology and Therapeutics (1996), 72, vol 1: 51-81).

Nicotine derivatives have been described for use in treating Parkinson's disease. Examples are U.S. Pat. Nos. 5,232,933, 5,242,935, 8,741,348, 8,003,080, 8,980,308, 7,718,677, 6,238,689, 6,911,475, International Application Publication No. WO 2012/101060.

Further, a study published in 1994 described results of chronic nicotine treatment in rats. Janson et al., “Chronic nicotine treatment counteracts dopamine D2 receptor upregulation induced by a partial meso-diencephalic hemitransection in the rat,” Brain Research. Volume 655. Issues 1-2, 29 Aug. 1994, pages 25-32.

Additionally, a new randomized, placebo-controlled study found “significant nicotine-associated improvements in attention, memory, and psychomotor speed,” with excellent safety and tolerability in patients with amnestic mild cognitive impairment. The study looks at this and other recent data suggesting that transdermal nicotine could be neuroprotective for neurological disorders. Neurology Today: 19 Jan. 2012—Volume 12—Issue 2—pp 37, 38, Hurley, Growing List of Positive Effects of Nicotine Seen in Neurodegenerative Disorders. Hurley, Dan.

A study published in 2012 involved 67 subjects with amnestic MCI randomized for six months to either placebo or 15 mg per day of transdermal nicotine. The results found “significant nicotine-associated improvements in attention, memory, and psychomotor speed,” with excellent safety and tolerability. Thus, the published benefits of nicotine therapy include the treatment of dyskinesia and impulsivity in Parkinson disease, cognitive defects in attention deficit-hyperactivity disorder (ADHD), and attention and memory in mild cognitive impairment (MCI). Newhouse, et al., “Nicotine treatment of mild cognitive impairment: a 6-month double-blind pilot clinical trial,” Neurology 2012 Jan. 10; 78(2):91-101.

New oral nicotine administration therapies have been suggested. A recent publication described the continuous or progressive administration of 0.2 mg to 5 mg per day per kilogram of body weight in man simultaneously with levodopa in a dose at least 30% lower than the effective dose when levodopa is administered alone (Mov Disord. 2012 July; 27(8): 947-957. Published online 2012 Jun. 12. “Nicotine as a potential neuroprotective agent for Parkinson's disease,” Quik et al. See also US Patent Publ. No. 2013/0017259A1).

None of the available nicotine treatments, however, provide consistent and effective doses for the treatment of Parkinson's and/or LID. Further, current drug delivery systems for the treatment of Parkinson's and/or LID are unable to deliver variable, patient individualized doses for each individual delivery each day, or can only do so with extreme patient compliance. Accordingly, the development of a drug and corresponding delivery system for Parkinson's that restores the functionality of D1 and D2 dopaminergic receptors remains a major problem in the field of neurodegenerative disease. A treatment that solves some or all of these problems is therefore desired.

### SUMMARY OF THE DISCLOSURE

Described herein is the programmable, variable, and patient-individualized transdermal administration of nicotine and/or combinations of nicotine with other drugs for the treatment of Parkinson's disease, levodopa induced dyskinesia (LID), multi-systematized atrophies, gait disorders and/or cognitive disorders (such as, e.g., Alzheimer's disease, attention deficit hyperactivity disorder, schizophrenia, and neurodegenerative diseases in general) using a wearable transdermal delivery device. In some embodiments, the wearable devices have one or more sensors, such as an accelerometer, to record and analyze movements and other bodily functions to improve the therapy delivered by the device. Some embodiments include a companion smart-phone application to improve patient compliance, quality of life, and cognitive function and/or to tailor the drug therapy to the patient's needs.

In particular, described herein are devices and methods for delivering nicotine transdermally to achieve a pharmacokinetic (PK) profile similar to that of oral nicotine therapy. For example, to the extent that the periodic oral administration of nicotine results in a blood/plasma concentration of nicotine that varies with time, the present invention provides a device and method to achieve similar blood/plasma concentration profiles.

In general, in one embodiment, a method of delivering nicotine to treat dyskinesia, such as Levodopa induced dyskinesia (LID), includes delivering a first dose of nicotine to a patient using a transdermal delivery device and delivering a second dose of nicotine to the patient using the transdermal delivery device. The first and second doses are timed such that a refractory period between peak plasma levels of nicotine in the patient prevents desensitization of nicotinic acetylcholine receptors while reducing symptoms of LID.

This and other embodiments can include one or more of the following features. The first and second doses can be delivered within 24 hours. The method can further include repeating the delivery of first and second doses for at least one month. A total amount of nicotine delivered in the 24 hours can be between 20 and 30 mg. Only two doses of nicotine can be delivered in the 24 hours so as to create a two-peak concentration profile over the 24 hours. The refractory period can be between 5 hours and 15 hours. The refractory period can be between 10 and 12 hours. The first dose can include multiple boluses administered within one hour. There can be three boluses, and each bolus can include between 70 and 80 μL of nicotine formulation. A peak to trough ratio at the refractory period can be between 10 and 80. A peak to trough ratio at the refractory period can be between 15 and 30. The symptoms of LID can be reduced by at least 30%. The symptoms of LID can include tremors, headache, changes in motor function, changes in mental status, changes in sensor functions, seizures, insomnia, paresthesia, and/or dizziness. The nicotine can be delivered as a combination therapy. The nicotine can be delivered as a combination therapy with amantadine, memantine, donepezil, levodopa, carbidopa, a dopamine agonist, apomorphine, rotigotine, rasagaline, an anticholinergic. MAO-B Inhibitors, COMT inhibitors, pramipexole, ropinirole, piribedil, cabergoline, lisuride, selegiline, bromocriptine, pergolide, or safinamide.

In general, in one embodiment, a method of treating dyskinesia, such as Levodopa induced dyskinesia (LID), includes: (1) delivering one or more first doses of nicotine to the patient according to an initial dosage protocol using a transdermal delivery device; (2) gathering data from a sensor of the transdermal delivery device, (3) analyzing the data to determine a severity of one or more symptoms of LID; (4) if the severity of symptoms is over a set amount, then increasing the dosage protocol; and (5) delivering one or more second doses according to the increased dosage protocol.

This and other embodiments can include one or more of the following features. The sensor can be an accelerometer, gyroscope, magnetometer, or barometric sensor. The method can further include repeating the steps of delivering, gathering, analyzing, and increasing so as to up-titrate the dosage of nicotine over time. The dosage can be up-titrated over a period of at least one month. Gathering data from a sensor can include gathering data with a companion application associated with the transdermal delivery device. The method can further include gathering user input from a companion application of the transdermal delivery device, analyzing the user input to determine a severity of one or more symptoms of LID based upon the user input, and delivering the one or more second doses according to the second increased dosage protocol. If the severity of the symptoms based upon the user input is over a set amount, then dosage protocol can be increased to a second increased dosage protocol. The symptoms of LID can include tremors, headache, changes in motor function, changes in mental status, changes in sensor functions, seizures, insomnia, paresthesia, and/or dizziness. The nicotine can be delivered as a combination therapy. The nicotine can be delivered as a combination therapy with amantadine, memantine, donepezil, levodopa, carbidopa, a dopamine agonist, apomorphine, rotigotine, rasagaline, an anticholinergic, MAO-B Inhibitors, COMT inhibitors, pramipexole, ropinirole, piribedil, cabergoline, lisuride, selegiline, bromocriptine, pergolide, or safinamide. The severity of the symptoms can be reduced by at least 10% when the nicotine is administered according to the increased dosage protocol rather than the initial dosage protocol.

In general, in one embodiment, a method of treating dyskinesia, such as Levodopa induced dyskinesia (LID), includes delivering one or more first doses of nicotine to the patient according to a dosage protocol using a transdermal delivery device. The dosage protocol can result in peak plasma levels in the patient at set peak times. The method also includes gathering data from a sensor of the transdermal delivery device, gathering data from a sensor of the transdermal delivery device, and analyzing the data to determine a timing of one or more symptoms of LID. If the timing of the one or more symptoms of LID is offset from the set peak times, then the method includes adjusting the dosage protocol so as to shift the set peak times to more closely overlap with the timing of the one or more symptoms.

This and other embodiments can include one or more of the following features. Analyzing the data to determine a timing of one or more symptoms of LID can include analyzing the data to determine a timing of a peak severity of symptoms during a 24 hour period. The dosage protocol can include a plurality of set peak times in 24 hours. There can be two set peak times in 24 hours. The sensor can be an accelerometer, gyroscope, magnetometer, or barometric sensor. Gathering data from a sensor can include gathering data with a companion application associated with the transdermal delivery device. The method can further include gathering user input from a companion application of the transdermal delivery device and analyzing the user input to determine a timing of one or more symptoms of LID based upon the user input. If the timing of the symptoms based upon the user input is offset from the set peak times, then the method can include adjusting the dosage protocol so as to shift the set peak times to more closely overlap with the timing of the one or more symptoms. The symptoms of LID can include tremors, headache, changes in motor function, changes in mental status, changes in sensor functions, seizures, insomnia, paresthesia, and/or dizziness. The nicotine can be delivered as a combination therapy. The nicotine can be delivered as a combination therapy with amantadine, memantine, donepezil, levodopa, carbidopa, a dopamine agonist, apomorphine, rotigotine, rasagaline, an anticholinergic, MAO-B Inhibitors, COMT inhibitors, pramipexole, ropinirole, piribedil, cabergoline, lisuride, selegiline, bromocriptine, pergolide, or safinamide.

In general, in one embodiment, a transdermal delivery device includes a formulation reservoir configured to hold a nicotine formulation therein, a transdermal membrane configured to deliver the nicotine formulation from the reservoir to the patient, and a sensor configured to detect one or more symptoms associated with Levodopa induced dyskinesia (LID).

This and other embodiments can include one or more of the following features. The device sensor can be an accelerometer, gyroscope, magnetometer, or barometric sensor. The symptoms of LID can include tremors, headache, changes in motor function, changes in mental status, changes in sensor functions, seizures, insomnia, paresthesia, and/or dizziness. The device further includes a controller that can be configured to gather data from the sensor, analyze the data to determine a severity of the one or more symptoms of LID, and if the severity of symptoms is over a set amount, then increase a dosage protocol for the nicotine formulation. The device further includes a controller that is configured to gather data from the sensor and analyze the data to determine a timing of the one or more symptoms of LID. If the timing of the one or more symptoms of LID is offset from peak nicotine concentration times in a patient using the transdermal delivery device, then the controller can adjust a dosage protocol for the nicotine formulation so as to shift the set peak times to more closely overlap with the timing of the one or more symptoms. The device can further include a dispensing mechanism that can be configured to deliver a plurality of boluses of nicotine formulation from the formulation reservoir to the transdermal membrane. The device can have structure enabling removal of solvent from the transdermal membrane. The device can further include a controller that is configured to deliver a first dose of the nicotine formulation to a patient using the transdermal delivery device and a second dose of nicotine to the patient. The first and second doses can be timed such that a refractory period between peak plasma levels of nicotine in the patient prevents desensitization of nicotinic acetylcholine receptors while reducing symptoms of LID.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows an exemplary transdermal drug delivery device.

FIG. 1B shows another exemplary transdermal drug delivery device.

FIG. 2 shows a simulated oral plasma concentration vs. time profile.

FIG. 3 shows clinical data for transdermal plasma concentration vs time.

FIG. 4 shows a two-compartmental pharmacokinetic model.

FIG. 5 shows a simulated oral and transdermal (one-peak) plasma concentration vs time profile.

FIG. 6 shows a simulated oral and transdermal (two-peak) plasma concentration vs time profile.

FIG. 7 shows a simulated oral and transdermal (three-peak) plasma concentration vs time profile.

FIG. 8 shows a flow chart for changing a drug delivery protocol to titrate up the drug dosage.

### DETAILED DESCRIPTION

Described herein are transdermal nicotine delivery devices and methods that can be used to treat or manage Parkinson's and/or LID. For example, the delivery devices and methods described herein can deliver nicotine in a pulsatile fashion to treat Parkinson's and/or LID. Further, the pulsatile delivery of nicotine described herein can be tuned to address symptom variations from patient to patient.

The devices and methods described herein may be particularly beneficial at the outset of treatment of Parkinson's and/or LID, as the delivered doses of nicotine can be titrated up. In addition, the amount of nicotine delivered for prolonged maintenance can advantageously be varied depending on the physiological characteristics of the patient. Pulsatile nicotine delivery as described herein can advantageously increase efficacy of the drug regimen and decrease the build-up of tolerance and desensitization. Further, the “refractory periods” between doses can lead to a cycle of receptor activation, desensitization, and re-sensitization, potentially lowering the amount of nicotine required to be effective compared to the amount of nicotine required to achieve the same therapeutic effect via continuous delivery.

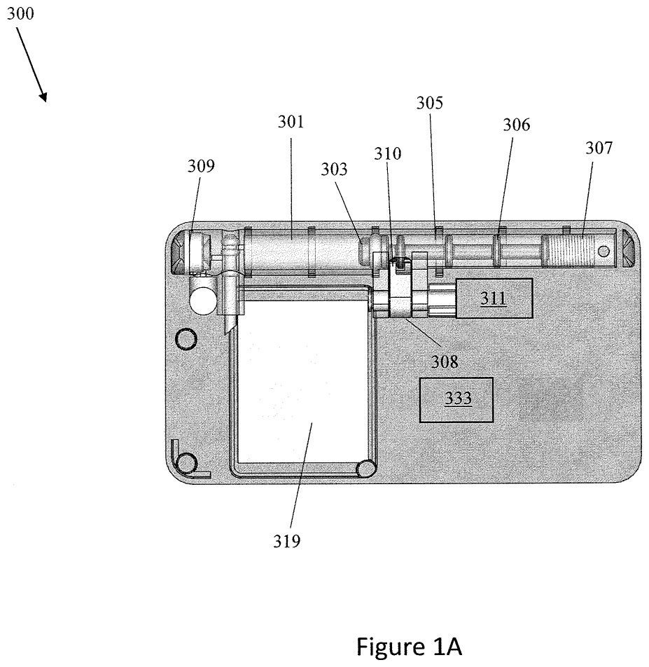

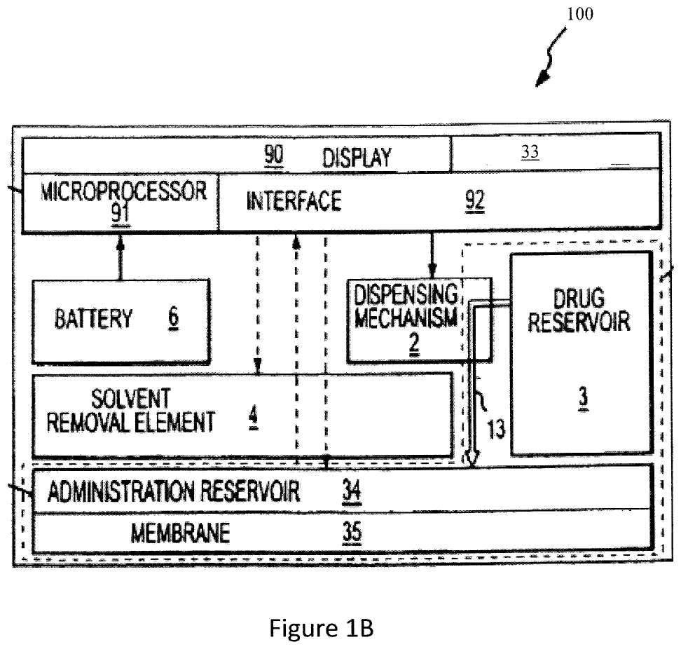

In some embodiments, the pulsatile nicotine delivery can be provided to a patient to treat Parkinson's and/or LID using a transdermal drug delivery device, such as the device shown in FIG. 1A. The transdermal delivery device 300 of FIG. 1A includes a reservoir 301. Further, a plunger including a piston 303 and control rod 305 can extend at least partially within the reservoir 301. A compressed spring 307 can bias the control rod 305 and piston 303 towards the reservoir 301. The control rod 305 can include a plurality of teeth 306 thereon. Further, a rotatable cam having two cam surfaces 310 can be positioned such that the cam surfaces 310 can engage with the teeth 306 of the control rod 305. The cam surfaces 310 can be semi-circular and can be circumferentially offset relative to one another (e.g., such that there is no circumferential overlap between the two surfaces 310). A valve 309, such as an umbrella valve, can be positioned at the distal end of the reservoir 301 and can prevent fluid from exiting the reservoir 301 until activated by the piston 303. Further, a motor 311 can be connected to the cam 308 so as to rotate the cam 308. A transdermal membrane 310 can be fluidically connected to the reservoir 301 so as to transfer fluid to the skin of the patient during use of the device 300. The device 300 can be configured such that activation of the motor 311 rotates the cam 308 so as to sequentially release the teeth 306 of the control rod 310, thereby providing for pulsatile delivery of fluid. Similar devices are described in PCT Application No. PCT/US18/12568, filed Jan. 5, 2018, titled “TRANSDERMAL DRUG DELIVERY DEVICES AND METHODS”, the entirety of which is incorporated by reference herein.

In some embodiments, the device 300 can further include one or more sensors 333, such as one or more of an accelerometer, compliance sensor, gyroscope, magnetometer, and/or barometric sensors. The one or more sensors 333 can be connected to a controller to provide real time and/or delayed feedback on the patient's movement and/or other conditions. In some embodiments, the sensor can be used to detect a characteristic associated with a symptom of PD or LID. For example, the symptom can be tremors, headache, changes in motor function, changes in mental status, changes in sensor functions, seizures, insomnia, paresthesia, and/or dizziness.

Another exemplary transdermal delivery device is shown in FIG. 1B. The device 100 includes an administration reservoir 34 positioned over a transdermal microporous membrane 35. The device 100 further includes a dispensing mechanism 2, such as a micropump, a formulation reservoir 3, a solvent removal element 4, and a battery 6. A liquid can be provided in the active substance reservoir 3 for dispensing via feed chamber or delivery tube 13. The liquid can include a sufficient or predetermined amount of one or more active substances dissolved or dispersed at an appropriate concentration in a formulation that contains a solvent (or more volatile liquid) or a mixture of solvent along with the active substances. For example, the solvent may include one or more generally regarded as safe (GRAS) agents such as water, ethanol and other low molecular weight alcohols, acetone, ethyl acetate, volatile oils or the like. The solvent removal element 4 can be provided in the device 100 to control dosing by removing the solvent or fluid mixture. The element 4 may include desiccant, absorbent material, or other material to absorb evaporating solvent, with the element 4 being connected to the administration element such as by one or more tubes (not shown). A connection is shown between the interface or drive circuit 92 of control unit 91, and this may be used to sense the concentration of a active substance in the administration reservoir 34 and to control operation of the solvent removal element (e.g., in embodiments where active components are provided to further solvent removal as discussed below). The device 100 can further include a display 90 and user interface 92. Additionally, the device 100 can include a gas permeable membrane therein. Similar devices are described in U.S. Publication No. US 2017-0224911 A1, titled “BIOSYNCHRONOUS TRANSDERMAL DRUG DELIVERY FOR LONGEVITY, ANTI-AGING, FATIGUE MANAGEMENT, OBESITY, WEIGHT LOSS, WEIGHT MANAGEMENT, DELIVERY OF NUTRACEUTICALS, AND THE TREATMENT OF HYPERGLYCEMIA, ALZHEIMER'S DISEASE, SLEEP DISORDERS, PARKINSON'S DISEASE, AIDS, EPILEPSY, ATTENTION DEFICIT DISORDER, NICOTINE ADDICTION, CANCER, HEADACHE AND PAIN CONTROL, ASTHMA, ANGINA, HYPERTENSION, DEPRESSION, COLD, FLU AND THE LIKE”, the entirety of which is incorporated by reference herein.

The device 100 can further include one or more sensors 33, such as one or more of an accelerometer, compliance sensor, gyroscope, magnetometer, and/or barometric sensors. The one or more sensors 33 can be connected to a controller to provide real time and/or delayed feedback on the patient's movement and other conditions. In some embodiments, the sensor can be used to detect a characteristic associated with a symptom of PD or LID. For example, the symptom can be tremors, headache, changes in motor function, changes in mental status, changes in sensor functions, seizures, insomnia, paresthesia, and/or dizziness.

In some embodiments, the device 300 or 100 can provide programmable, variable, tunable and patient-individualized transdermal delivery of nicotine from the reservoir. The nicotine can be provided either alone or in combination, for example, with amantadine, memantine, donepezil, levodopa, or carbidopa. The transdermal delivery device 300 or 100 can be used, for example, to supply the nicotine and/or combination nicotine formulation in an optimal temporal pulsatile pattern to help prevent adverse the side effects of LID while ensuring increased efficacy of the Parkinson's and/or LID treatment.

In some embodiments, the device 300 or 100 can be used with an associated smart phone application and/or sensor technology to help ensure that the active ingredient (e.g., nicotine) is administered to, and is bioavailable to, the patient according to the most optimal desired temporal pattern and in the most optimal dosages.

Further, in some embodiments, the device 300 or 100 can include two separable parts, including a cartridge and a control unit The cartridge unit can, for example, be disposable while the control unit can, for example, be reusable. The cartridge and the control unit can require limited force to attach or detach, which can also be beneficial for patients with Parkinson's.

In some embodiments, the device 300 or 100 can include a user interface on a cell phone, console or a PC for ease of use. This can be beneficial for Parkinson's disease patients, which have limited dexterity in their hands.

Parkinson's disease patients tend to be non-smokers, and administration of nicotine (with or without a nicotine companion drug) in patients who have not previously used nicotine can cause them to experience severe or unpleasant side effects. Dosages may therefore be carefully and gradually titrated up for each patient to prevent adverse side effects associated with nicotine use. Further, with respect to not just the titration period, but to the entire duration of treatment, certain patients can metabolize nicotine more quickly, meaning they can better handle larger doses quickly or require higher dosages throughout the treatment period. Conversely, slower metabolizers require a slower and more gradual increase in nicotine to avoid adverse side effects and less nicotine to be effective throughout the duration of the therapy. Each patient may need his/her own specific dosing and timing regimen for titrating up and prolonged maintenance of the nicotine administration. Accordingly, in some embodiments, the formulation (e.g., nicotine and/or a nicotine companion drug) in the reservoir can be administered in accordance with a protocol that includes gradually increasing the amount of nicotine and requisite maintenance doses of nicotine or its derivatives over a first period that extends from one day to 24 months, followed by a second period of a patient-specific dosage schedule, such as stabilized doses, that can occur over the following months or years. In some embodiments, the increase in the dose of nicotine over the first period can be accompanied by a concomitant reduction in an amount of companion drug, such as levodopa, as improvements in Parkinson's are observed.

In some embodiments, the transdermal delivery devices described herein can deliver pulsatile nicotine that mimics the PK profile of orally administered nicotine.

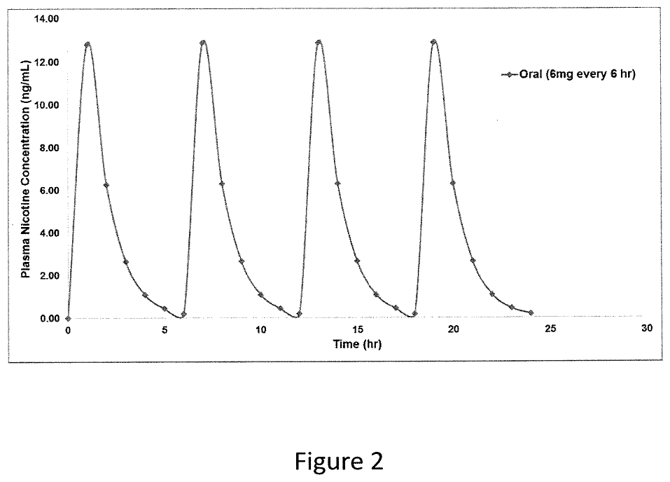

An exemplary graph of plasma nicotine concentration versus time for oral administration of nicotine to a human subject is shown in FIG. 2. The graph is based on the oral nicotine administration protocol administering a 6 mg nicotine tablet every 6 hours, as described in International Patent Publication No. WO 2013/006643, the entirety of which is incorporated by reference herein.

To obtain the graph shown in FIG. 2, the plasma concentration-time profile of oral nicotine was simulated using the pharmacokinetic equation (equation 1) for multiple-dose oral administration:

$${C}_{P} = \frac{F \times {D}_{S} \times {k}_{a}}{{V}_{D} ( {k}_{a} - {k}_{e} )} ( [ \frac{1 - {e}^{- n {k}_{e} τ}}{1 - {e}^{- {k}_{e} τ}} ] {e}^{- {k}_{e} t} - [ \frac{1 - {e}^{- n {k}_{a} τ}}{1 - {e}^{- {k}_{a} τ}} ] {e}^{- {k}_{a} t} ) \qquad ( equation 1 )$$

where F is oral bioavailability. D is oral dose, kₐ is absorption rate constant. V_{d} is volume of distribution, kₑ is elimination rate constant, s is dose interval, n is the number of dose, and C is the plasma concentration at any time ‘t’. The pharmacokinetic parameters used for the simulation of FIG. 2 are listed in the table below:

| Parameters | Average Values | Range |
|---|---|---|
| F | 0.32 | 0.20-0.44 |
| D (mg) | 6 | — |
| kₐ (hr−1) | 2.5 | — |
| V_{d} (L) | 76 | 33-119 |
| kₑ (hr−1) | 0.9 | 0.85-0.95 |
| τ (Tau) | 6 | |

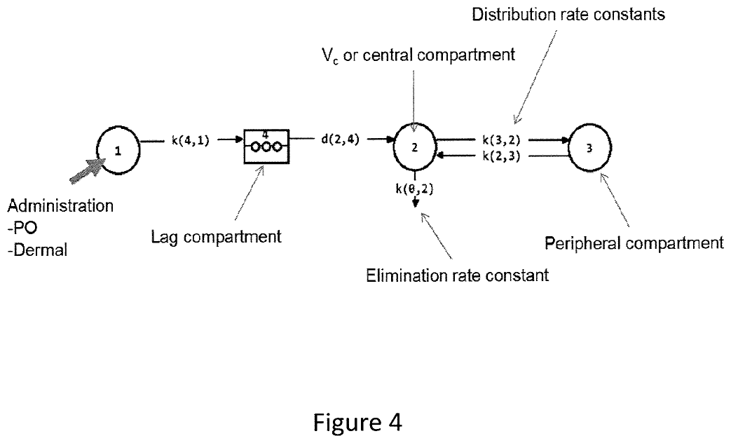

The pharmacokinetic parameters F, D, and T were obtained from Hukkanen, J. et al. Pharmacol Rev. 2005 March; 57(1):79-115 and U.S. Patent Publication No. 20130017259A1. Further, the pharmacokinetic parameter absorption rate constant (kₐ) shown in the above table was obtained by fitting the orally administered nicotine PK data (obtained from Benowitz, N. L. et al. Clin Pharmacol Ther. 1991 March; 49(3):270-7) to a two-compartmental pharmacokinetic model (shown in FIG. 4). Pharmacokinetic parameters V_{d} (volume of distribution) and kₑ (elimination rate constant) were obtained by fitting intravenously administered nicotine PK data (obtained from Benowitz, N. L. et al. Clin Pharmacol Ther. 1991 March; 49(3):270-7) to the two-compartmental model shown in FIG. 4 without the absorption component and lag component.

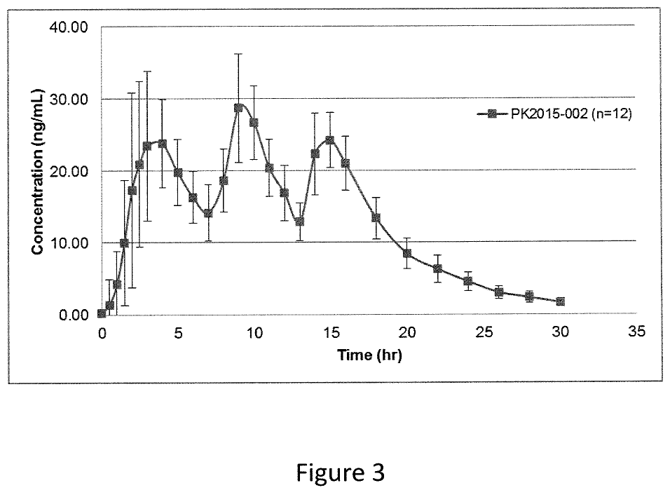

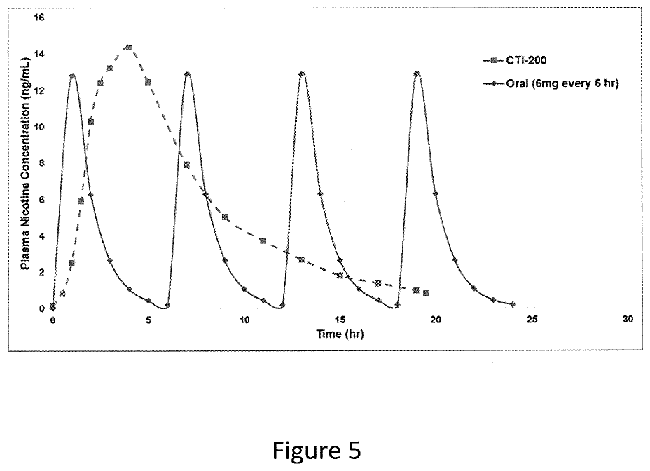

Exemplary graphs of plasma nicotine concentration versus time for transdermal administration of nicotine (dotted lines) to a human subject using a transdermal device as described herein relative to oral administration of nicotine (solid lines) are shown in FIGS. 5-7. To obtain the graphs shown in FIGS. 5-7, the plasma concentration-time profile of nicotine administered transdermally was simulated by means of a two compartmental model, as shown in FIG. 4. In addition to the two compartments (Central and peripheral), the lag compartment was included to take absorption lag into account associated with transdermal delivery. The pharmacokinetic parameters used for the simulation of FIGS. 5-7 are listed in the table below and were obtained by fitting the PK clinical data (shown in FIG. 3) for a device as described herein the two compartmental model of FIG. 4:

| Parameters | Average Values | Range |
|---|---|---|
| kₐ (hr−1) | 0.29 | 0.20-0.38 |
| V_{d} (L) | 59 | 13-105 |
| kₑ (hr−1) | 2.15 | 0.56-3.74 |

FIG. 5 shows a one-peak profile of nicotine delivery over a 24-hour period using a transdermal delivery system as described herein. The nicotine was a 5.4% w/v nicotine in 50:50 water:ethanol solution. Three boluses of 74 μL each were administered at 0 hours, 0.5 hours, and 1 hour for a total dose of 11.9 mg of nicotine.

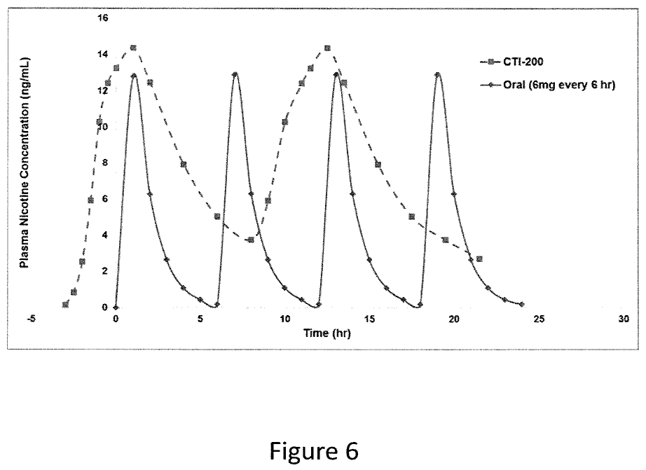

FIG. 6 shows a two-peak profile of delivery over a 24-hour period. The nicotine was a 5.4% w/v nicotine in 50:50 water:ethanol solution. Three boluses of 74 μL each were administered at 0 hours, 0.5 hours, and 1 hour and then again at 10.5 hours, 11 hours, and 11.5 hours for a total dosage of 23.9 mg of nicotine.

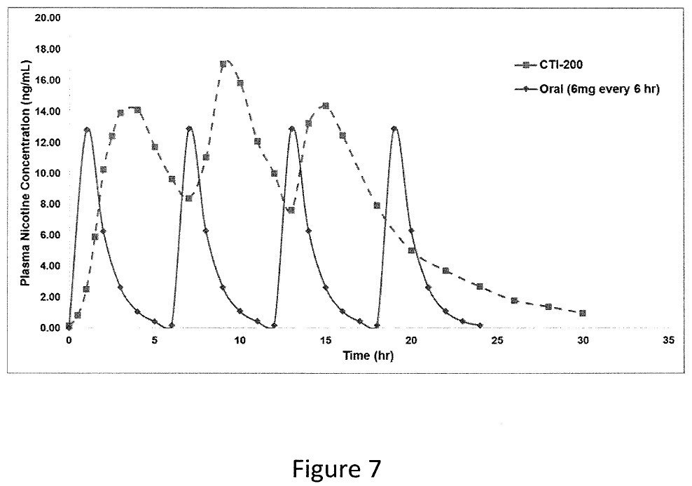

FIG. 7 shows a three-peak profile of delivery over a 24-hour period. The nicotine was a 5.4% w/v nicotine in 50:50 water:ethanol solution. Three boluses of 74 μL each were administered at 0 hours, 0.5 hours, and 1 hour, two boluses of 74 μL each were administered at 7 and 7.5 hours, and 1 bolus of 74 μL was administered at 13 hours for a total dosage of 23.9 mg of nicotine.

As shown in FIG. 6, the-two peak PK profile results in refractory periods between peak plasma levels comparable to the oral PK profile. The refractory periods, or time between the peak levels, can help prevent desensitization of nicotinic receptors. The refractory periods are demonstrated by peak-to-trough ratio. The one-peak PK profile of FIG. 5 and the three-peak PK profile of FIG. 7 can also provide the same maximum nicotine concentration, but with decreasing peak-to-trough ratios. In some embodiments, the refractory periods are between 5 and 15 hours, such as between 10 and 12 hours. In some embodiments, the peak-to-trough ratio for transdermal delivery of nicotine as described herein can be between 10 and 80, such as between 15 and 30. Further, time variations of plasma nicotine concentrations resulting from the transdermal administration of nicotine to a human subject using the drug delivery systems described herein can range between 5 ng/mL to 70 ng/mL, depending on the concentration of nicotine in the solution employed by the transdermal drug delivery device.

The transdermal delivery devices and methods described here can thus provide pulsatile drug delivery, allowing for customizable plasma drug concentration profiles with single or multiple peaks and troughs (refractory periods). The refractory periods may lead to a cycle of receptor activation, desensitization, and re-sensitization, lowering the effective nicotine dose compared to continuous delivery.

In some embodiments, the first dose of nicotine can be delivered prior to the patients' wake-up time, such as 3 hours prior to wake-up, in order to obtain a peak concentration upon, or shortly after, wake-up.

Parkinson's disease is a progressive neurodegenerative disorder, and each person reacts differently over time. Progress of the disease typically results in narrowing of the therapeutic window for widely prescribed drugs, such as levodopa. In some embodiments, the one or more sensors on the transdermal delivery devices descried herein can be used to understand exactly when a dyskinesia or other symptom is occurring and thus provide real-time drug protocol modification. In some embodiments, the one or more sensors can provide real-time feedback for contemporary protocol modification. For example, data from the one or more sensors can be used by a doctor to extend or modify a progressive drug protocol. In some embodiments, algorithmic artificially intelligent drug protocol modification and companion application modification can be employed based on the “smart” analysis of the patient's symptoms and circadian patterns. In such a “smart” analysis, a controller of the transdermal drug delivery systems can gather patient data from the one or more sensors over time through tracking, perform an algorithmic prediction based on patient-gathered information from the sensor, and/or carefully modify the drug delivery profile. For example, future intra-day drug protocols can be modified to match a circadian rhythm pattern. As another example, a dose adjustment can be made over time.

One exemplary method of modifying a transdermal drug delivery protocol (e.g., for the administration of nicotine to treat LID and/or PD) includes first collecting data from the one or more sensors and sending the data to a remote location, such as a server, cell phone, or personal computer. The data can include real time physiologic responses or real time data collection regarding LID or PD events and/or the current delivery protocol. In one embodiment, the data can be analyzed by a caregiver or health care professional, who can then change the protocol in response to an adverse event (e.g., severe symptoms) or non-compliance event by providing feedback to the user to change the drug protocol or inputting a change in protocol directly to the transdermal delivery device. In another embodiment, the data can be analyzed by an algorithmic computerized system that can send recommendations to the user regarding how to change the protocol, send recommendations to a caregiver or health care professional to modify the protocol, or automatically change the protocol in real-time or over time.

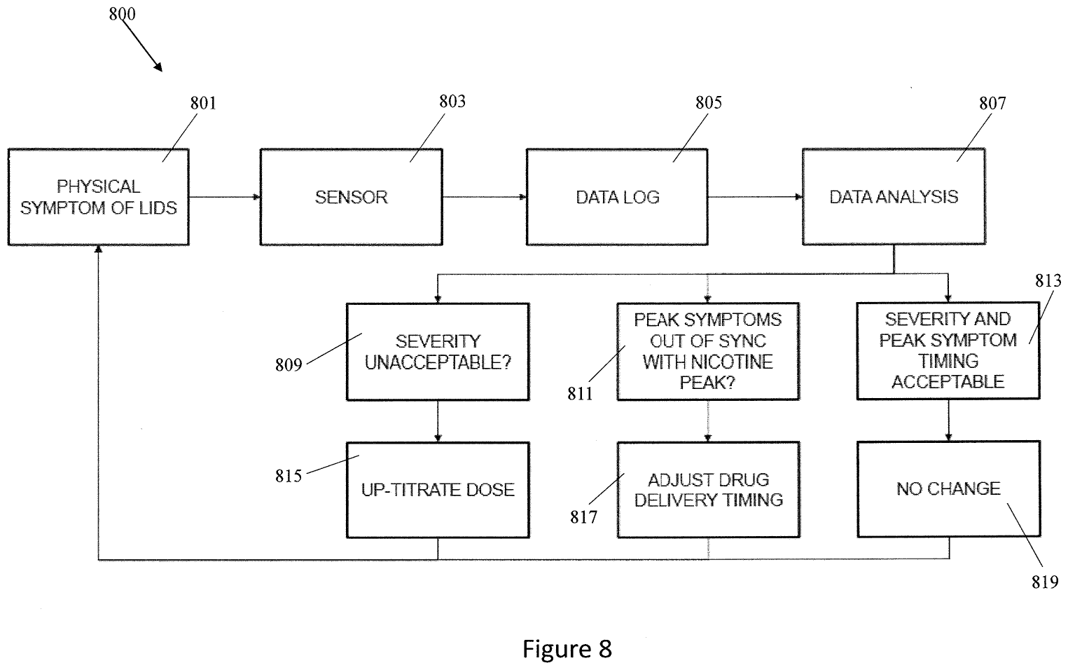

An exemplary flow chart 800 for changing the drug delivery protocol to titrate up the dosage is shown in FIG. 8. In most Parkinson's patients with LID who are non-smokers, the nicotine doses required for a therapeutic effect are likely to cause significant side effects, such as nausea, vomiting, and/or dizziness. Accordingly, the dosage of nicotine can be up-titrated to the lowest possible dose that achieves symptomatic relief while minimizing side effects. Thus, referring to FIG. 8, a patient can exhibit physical symptoms of LID (e.g., tremors) at step 801. At step 803, the one or more sensors on the transdermal delivery device can detect the symptoms. At step 805, a data log can be created from data gathered by the sensors. At step 807, the data can be analyzed. At step 809, if the severity of the symptoms (e.g., symptoms of LID) is unacceptable, then the dose can be up-titrated at step 815. At step 811, if the peak symptoms are out of sync with the nicotine peak, then the drug delivery timing can be adjusted at step 817 such that the peak nicotine concentration in plasma more closely aligns with the determined peak severity of symptoms. At step 813, if the severity and peak symptom timing is acceptable, then no change can be made at step 819. The process can then be repeated. In some embodiments, the dosage can be up-titrated in stages (e.g., over days or weeks), the patient's response can be monitored, and then up-titration and/or modification of the dose timing can be continued.

In some embodiments, data obtained by the sensors can be stored on the device. In other embodiments, the data can be stored in the application. In other embodiments, the data can be stored on a remote service (e.g., cloud server).

In some embodiments, the transdermal drug delivery systems described herein can be paired with a companion application (e.g., for a smart phone, laptop, or computer) to assist non-pharmacokinetic aspects of therapy. In some embodiments, the companion application can lend a holistic approach to the treatment, for example, providing additional support to improve quality of life in the form of meditation, yoga, singing, music, dance, and/or exercise recommendations. The application can include puzzles and/or games for cognitive improvement. The application can remind the patient to take one or more medications. The application can provide a platform for patients to record details about their diet, such as a description and timing of meals, which may be important because food may affect the absorption pattern of certain medications, e.g., levodopa. The application may include information about food recipes, for example, that have been shown to be beneficial for Parkinson's disease patients. The application may be used for alerting a patient's relatives and/or physician in case of emergency or non-compliance.

Further, as described above, in some embodiments, the transdermal drug delivery device can include one or more sensors, such as one or more of an accelerometer, compliance sensor, gyroscope, magnetometer, and/or barometric sensors, to provide real time and/or delayed feedback on the patient's movement and other conditions. The companion application can gather information from the one or more sensors to assist, for example, in cognitive therapy, drug therapy, data gathering, compliance monitoring, and/or LID and other PD symptom patterns.

In some embodiments, the sensors and/or companion application described herein can be used to provide real-time compliance monitoring to alert doctors and caregivers if the patient isn't taking drug on time. Further, in some embodiments, the sensors and/or companion application can be used to provide information about symptom increases based on non-compliance. In some embodiments, user input into the application can be used to provide information about symptoms.

In some embodiments, the companion application can be used to gather data regarding a patient's symptoms of PD or LID. For example, the symptoms can be tremors, headache, changes in motor function, changes in mental status, changes in sensor functions, seizures, insomnia, paresthesia, and/or dizziness.

In some embodiments, algorithmic artificially intelligent companion application modification can be employed based on the “smart” analysis of the patient's symptoms and circadian patterns. In such a “smart” analysis, a controller of the transdermal drug delivery systems can gather patient data from the one or more sensors over time through tracking, perform an algorithmic prediction based on patient-gathered information from the sensor and the companion app, and/or carefully modify the drug delivery profile.

One exemplary method of modifying the companion application protocol is as follows. To begin, data can be collected from the application, such as results of games played, diet (e.g., as entered by the patient), and/or information gathered from meditation, singing, or dancing performed with the application. In some embodiments, the data can include information about which functions the patient actively uses in the application, what content is accessed in the application, and/or how long the application is used. In some embodiments, the data can include any real time physiologic responses or real time data regarding LID or PD events, dosage protocol, and/or patient compliance data. The data can then be analyzed directly from the application or sent to a remote location, such as a server, cell phone, or personal computer. In some embodiments, the data can be analyzed by a caregiver or health care professional, who can then directly modify the application protocol, can make recommendations to the user to change the companion application inputs, or can make recommendations to the user to change the application protocol. In other embodiments, the data can be analyzed by an algorithmic computerized system to send recommendations to the user regarding how to change the application protocol, to make recommendations to the caregiver or health care professional regarding how to modify the application protocol, or can automatically change the application protocol in real-time or over time.

In some embodiments, the transdermal drug delivery devices described herein can include a combination therapy formulation in the drug reservoir.

Combination therapy or polytherapy to treat Parkinson's and LID is a therapy that uses more than one medication or modality (versus monotherapy, which is any therapy taken alone). Typically, these terms refer to using multiple therapies to treat a single disease, and often all the therapies are pharmaceutical (although it can also involve non-medical therapy, such as the combination of medications and talk therapy to treat depression). Pharmaceutical combination therapy may be achieved by prescribing/administering separate drugs, or, where available, dosage forms that contain more than one active ingredient (such as fixed-dose combinations) to treat multiple forms of symptoms manifesting from Parkinson's and LID or may be used together to combat one symptom.

In some embodiments, the combination therapies are contained in a single reservoir. In other embodiments, multiple reservoirs can be used. A transdermal drug delivery device with multiple reservoirs is described in PCT Application No. PCT/US17/64765, filed Dec. 5, 2017, tided, “TRANSDERMAL DRUG DELIVERY DEVICES AND METHODS”, the entirety of which is incorporated by reference herein.

In some embodiments, the transdermal drug delivery device can be configured to administer different dosages of each drug at different times, thereby advantageously allowing the device to be used to tune and individualize therapies with great precision.

In some embodiments, the drug delivery device described herein can use the one or more sensors and/or the companion application to gather data to modify the regular or combination therapy based on data emerging from patterns in symptoms. In some embodiments, the one or more sensors and/or the companion application can be configured to use the gathered data in algorithms to predict optimal combination therapy.

The transdermal drug delivery devices described herein can include, or be configured to be used with, a combination therapy that includes nicotine in combination with one or more other drugs such as amantadine, memantine, donepezil, levodopa, carbidopa, any other dopamine agonist, apomorphine, rotigotine or rasagaline, and other anticholinergics, MAO-B Inhibitors or COMT inhibitors, pramipexole, ropinirole, piribedil, cabergoline, lisuride, selegiline, bromocriptine, pergolide, or safinamide. In some embodiments, the combination therapy can include any combination of the above drugs.

In some embodiments, multiple strengths of any of the formulations described herein may be used with the transdermal device and/or methods described herein. In some embodiments, varying concentrations of nicotine in ethanol:water (1:1) can be used. The devices and methods described herein can be designed, for example, to deliver daily doses of nicotine from 3.5 mg up to 42 mg. Other doses of the other drugs can be used so as to fall within their therapeutic profile as appropriate. Examples of daily doses and corresponding nicotine concentrations that can be used are listed in the table below:

| Nicotine Concentration | Nicotine Dose |
|---|---|
| (% w/v) | (mg) |
| 0.9 | 3.5 |
| 1.8 | 7.0 |
| 2.7 | 10.5 |
| 3.6 | 14.0 |
| 4.5 | 17.5 |
| 5.4 | 21.0 |
| 10.8 | 42.0 |

In some embodiments, the transdermal delivery devices described herein can include an adhesive for adhesion to the patient's skin. The adhesive can be a reusable adhesive or a disposable/replaceable adhesive. In some embodiments, a strap can be used to hold the transdermal delivery device in place in lieu of or in addition to the adhesive.

In some embodiments, the transdermal delivery devices described herein can be less than 15 mm in height.

In some embodiments, the transdermal delivery devices described herein can include a user interface, such as a screen, that allows for haptic feedback. The feedback may be used, for example, to increase the level of compliance in patients by reminding them to take their other medication (e.g., levodopa) as per pre-programmed schedule and/or to change the drug delivery schedule.

In some embodiments, a nesting/docking station can be used for assembling a two-part drug delivery devices as described herein. The nesting/docking station can also be used as a console for charging the device (e.g., for charging a rechargeable battery of the device). The nesting/docking station can further include a screen for setting a delivery time remotely. Additionally, the nesting/docking station can be configured to connect to the internet to, for example, export data to the cloud while the transdermal delivery device is charged.

In some embodiments, the transdermal delivery devices described herein can be charged via modular charging using a battery pack. In other embodiments, the transdermal delivery devices described herein can have a disposable battery.

In some embodiments, the transdermal delivery devices described herein can be made available by prescription only.

In some embodiments, patients using the transdermal delivery devices described herein can be required to visit their caregiver regularly (e.g., every few months) to provide the caregiver with data collected from the one or more sensors.

The delivery devices and methods described herein can advantageously reduce the symptoms of Levodopa induced dyskinesia. For example, the symptoms of LID can be reduced by at least 30%, such as at least 40% or at least 50%.

The pulsatile transdermal administration of nicotine (with or without a nicotine companion drug) as described herein is in contrast to the use of instantaneous release systems, delayed release systems, or sustained release systems. Instantaneous release refers to systems that make the active ingredient available immediately after administration to the patient, such as via continuous or pulsed intravenous infusion or injections. Instantaneous release systems provide a great deal of control because administration can be both instantaneously started and stopped, and the delivery rate can be controlled with great precision. However, the administration is undesirably invasive as they involve administration via a puncture needle or catheter. Delayed release systems are systems in which the active ingredient is made available to the patient at some time after administration. Such systems include oral as well as injectable drugs in which the active ingredient is coated or encapsulated with a substance that dissolves at a known rate so as to release the active ingredient after a delay. Unfortunately, it is often difficult to control the degradation of the coating or encapsulant after administration, and the actual performance of the system will vary from patient to patient. Further, patient compliance necessary to achieve the dosage profiles desired for PD and LID patients are cumbersome and require a level of diligence that will likely not be met outside of a clinic. Finally, sustained release generally refers to release of active ingredient such that the level of active ingredient available to the patient is maintained at some level over a period of time. Like delayed release systems, sustained release systems are difficult to control and exhibit variability from patient to patient. Due to the adsorption through the gastrointestinal tract, drug concentrations in the patients' blood rise quickly in the body when taking a pill, but the subsequent decrease in blood concentrations is dependent on excretion and metabolism, which cannot be controlled. In addition, the adsorption through the gastrointestinal tract in many cases leads to considerable side effects (such as ulcers) and can severely damage the liver.

The transdermal drug delivery devices and methods described herein can be used to treat Parkinson's, LID, neurological disorders arising from trauma, ischemic or hypoxic conditions that can be treated include stroke, hypoglycemia, cerebral ischemia, cardiac arrest, spinal cord trauma, head trauma, perinatal hypoxia, cardiac arrest and hypoglycemic neuronal damage; neurodegenerative disorders such as epilepsy, multiple sclerosis, Alzheimer's disease, Huntington's disease. Parkinsonism, and amyotrophic lateral sclerosis; other diseases or disorders such as convulsion, pain, depression, anxiety, schizophrenia, muscle spasms, migraine headaches, urinary incontinence, nicotine withdrawal, opiate tolerance and withdrawal, emesis, brain edema, tardive dyskinesia, AIDS-induced dementia, ocular damage, retinopathy, cognitive disorders, and/or neuronal injury associated with HIV-infection such as dysfunction in cognition, movement and sensation.

Any feature or element described herein with respect to one embodiment can be combined with, or substituted for, any feature or element described with respect to another embodiment. Further, transdermal drug delivery systems are described in US 2016/0220798 titled “Drug Delivery Methods and Systems,” the entirety of which is incorporated by reference herein in its entirety. Any feature or element described with respect to an embodiment herein can be combined with, or substituted for, any feature or element described in US 2016/0220798.

In some embodiments, the methods and/or devices described herein can be used for non-transdermal drug delivery methods, including oral, nasal, buccal, or subcutaneous or other.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”. “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of delivering nicotine to treat Levodopa induced dyskinesia (LID), the method comprising:
delivering a first dose of nicotine to a patient using a transdermal delivery device; and delivering a second dose of nicotine to the patient using the transdermal delivery device, the first and second doses timed such that a refractory period between peak plasma levels of nicotine in the patient prevents desensitization of nicotinic acetylcholine receptors while reducing symptoms of LID.

2. The method of claim 1, wherein the first and second doses are delivered within 24 hours.

3. The method of claim 2, further comprising repeating the delivery of first and second doses for at least one month.

4. The method of claim 2, wherein a total amount of nicotine delivered in the 24 hours is between 20 and 30 mg.

5. The method of claim 2, wherein only two doses of nicotine are delivered in the 24 hours so as to create a two-peak concentration profile over the 24 hours.

6. The method of claim 1, wherein the refractory period is between 5 hours and 15 hours.

7. The method of claim 1, wherein the refractory period is between 10 and 12 hours.

8. The method of claim 1, wherein the first dose includes multiple boluses administered within one hour.

9. The method of claim 8, wherein there are three boluses, and wherein each bolus includes between 70 and 80 μL of nicotine formulation.

10. The method of claim 1, wherein a peak to trough ratio at the refractory period is between 10 and 80.

11. The method of claim 1, wherein a peak to trough ratio at the refractory period is between 15 and 30.

12. The method of claim 1, wherein the symptoms of LID are reduced by at least 30%.

13. The method of claim 1, wherein the symptoms of LID include tremors, headache, changes in motor function, changes in mental status, changes in sensor functions, seizures, insomnia, paresthesia, and/or dizziness.

14. The method of claim 1, wherein the nicotine is delivered as a combination therapy.

15. The method of claim 14, wherein the nicotine is delivered as a combination therapy with amantadine, memantine, donepezil, levodopa or carbidopa, any other dopamine agonist, apomorphine, rotigotine, rasagaline, anticholinergics, MAO-B Inhibitors, COMT inhibitors, pramipexole, ropinirole, piribedil, cabergoline, lisuride, selegiline, bromocriptine, pergolide, or safinamide.

16.-35. (canceled)

36. A transdermal delivery device comprising:
a formulation reservoir configured to hold a nicotine formulation therein;
a transdermal membrane configured to deliver the nicotine formulation from the reservoir to the patient; and
a sensor configured to detect one or more symptoms associated with Levodopa induced dyskinesia (LID).

37. The device of claim 36, wherein the sensor is an accelerometer, gyroscope, magnetometer, or barometric sensor.

38. The device of claim 36, wherein the symptoms of LID include tremors, headache, changes in motor function, changes in mental status, changes in sensor functions, seizures, insomnia, paresthesia, and/or dizziness.

39. The device of claim 36, further comprising a controller, the controller configured to:
gather data from the sensor;
analyze the data to determine a severity of the one or more symptoms of LID; and
if the severity of symptoms is over a set amount, then increase a dosage protocol for the nicotine formulation.

40. The device of claim 36, further comprising a controller, the controller configured to:
gather data from the sensor;
analyze the data to determine a timing of the one or more symptoms of LID;
if the timing of the one or more symptoms of LID is offset from peak nicotine concentration times in a patient using the transdermal delivery device, then adjust a dosage protocol for the nicotine formulation so as to shift the set peak times to more closely overlap with the timing of the one or more symptoms.

41. The device of claim 36, further comprising a dispensing mechanism configured to deliver a plurality of boluses of nicotine formulation from the formulation reservoir to the transdermal membrane.

42. The device of claim 36, wherein the device has structure enabling removal of solvent from the transdermal membrane.

43. The device of claim 36, further comprising a controller, the controller configured to:
deliver a first dose of the nicotine formulation to a patient using the transdermal delivery device; and
deliver a second dose of nicotine to the patient, the first and second doses timed such that a refractory period between peak plasma levels of nicotine in the patient prevents desensitization of nicotinic acetylcholine receptors while reducing symptoms of LID.
